(19) United States
(12) Patent Application Publication
(10) Pub. No.: US 2007/0144511 A1
(43) Pub. Date: Jun. 28, 2007

(54) Nebulizer and method therefor

(76) Inventors: Anders Reden, Stockholm (SE); Mattias Mikaelsson, Sundbyberg (SE)

Correspondence Address: FASTH LAW OFFICES (ROLF FASTH), 26 PINECREST PLAZA, SUITE 2, SOUTHERN PINES, NC, 28387-4301, US

(21) Appl. No.: 10/598,812

(22) PCT Filed: Mar. 2, 2005
(86) PCT No.: PCT/US05/06624

§ 371 Date: Sep. 12, 2006

Related U.S. Application Data
(60) Provisional application No. 60/557,274, filed on Mar. 30, 2004.

(51) Int. Cl. A61M 11/00; A61M 16/00

(52) U.S. Cl. 128/200.14; 128/204.18

(57) ABSTRACT

The method is for nebulizing a patient. A nebulizer is provided that has an upper semi-spherical housing in operative engagement with a lower semi-spherical housing. The upper housing has openings to receive a tube that has an upward opening defined therein. The nebulizer is connected to a respiration system downstream of incoming air of an inhale tube and upstream of a connector. The pressurized air is added in a tube connected to a bottom of the housing. The air is exposed to a liquid medication disposed in the housing. The air aero-soles the liquid medication into an aero-soled medication. The aero-soled medication mixes with inhaling air provided by a ventilator unit and moisturized by an upstream filter. The aero-soled medication flows into the opening of the tube and then into a patient.

### TECHNICAL FIELD

The present invention relates to a nebulizer and a method of using same.

### BACKGROUND OF THE INVENTION

Nebulization is performed on almost all ventilator dependent patients. It is common to nebulize patients about 4-6 times every 24 hours. With today's technology, the patients must be disconnected from the life sustaining respirator during a short time when a heat-moist exchanger (HME) is temporarily removed and the nebulizer is applied to the tubes of the respirator. At the end of the nebulization process, it is again necessary to disconnect the patient from the respirator to reconnect the HME and, if necessary, to clean the nebulizer.

The nebulization and the disconnection from the ventilator may take between 15-30 seconds. During this time, the patient is not provided with the life sustaining air and oxygen. The lungs may collapse and for very sick patients this may result in an acute loss of SaO2 (saturation level of oxygen in the blood) and that there is a delay until the adequate lung pressure can be restored. Another problem of the prior art nebulizers is that there is a long distance between the nebulizer and the tube end inside the patient so that a substantial amount of medication gets caught in the tube and never reaches the lungs. It would be desirable to be able to do the nebulization closer to the lungs of the patient and without having to disconnect the air supply from the patient during nebulization. In other words, it would be desirable to administer the medicine to the patient without having to disconnect the life sustaining ventilator unit and to prevent the patient from inhaling. There is a need for a more effective nebulization system that does not have the drawbacks outlined above.

### SUMMARY OF THE INVENTION

The nebulizer of the present invention provides a solution to the above-outlined problems. More particularly, the method of the present invention is for using a unique nebulizer for nebulizing a patient. One important feature of the nebulizer is that it is fully functional even if it is turned 45 degrees relative to the horizontal plane. The nebulizer has an upper semi-spherical housing in operative engagement with a lower semi-spherical housing. The upper housing has openings to receive a tube that has an upward opening defined therein. The nebulizer is connected to a ventilator system downstream of incoming air of an inhale tube and upstream of a connector. Pressurized air is added in a tube connected to a bottom of the housing. The compressed air is introduced from the bottom of the tube and then allowed to expand at the jet hole. The resulting pressure courses fluid to be sucked up through the outer circular liquid feed tube. The air is exposed to a liquid medication disposed in the housing. The air aerosolizes the liquid medication into an aero-soled medication. The aero-soled medication mixes with inhaling air provided by a ventilator unit and moisturized by an upstream filter. The aero-soled medication flows into the opening of the tube and then into a patient.

By using the nebulizer of the present invention there is less risk that the lungs collapse and that the SaO2 content is reduced. Also, a bigger portion of the medicine reaches the patient's lungs. There is less risk of infection due to the more closed system and it is possible to use the nebulizer in combination with a closed suction system that is optimal for infection sensitive patients. It is not necessary to disturb the patient, particularly during the night, when it is time for nebulization.

### BRIEF DESCRIPTION OF THE DRAWING

FIG. 1 is a schematic side view of a prior art respiration system;

FIG. 2 is a schematic side view of the respiration system with a prior art nebulizer connected thereto;

FIG. 3 is a schematic side of the respiration system with the nebulizer of the present invention connected thereto;

FIG. 4 is an exploded view of the nebulizer of the present invention;

FIG. 5 is a cross-sectional view of the nebulizer of the present invention;

FIG. 6 is a cross-sectional top view of the nebulizer of the present invention;

FIG. 7 is a detailed view of the nebulizer at a 40 degree angle;

FIG. 8 is a cross-sectional side view of the tube of the nebulizer of the present invention;

FIG. 9 is an exploded view of an alternative embodiment of the present invention;

FIG. 10 is a cross-sectional view of the alternative embodiment shown in FIG. 9; and

FIG. 11 is a cross-sectional top view of the alternative embodiment shown in FIG. 9.

### DETAILED DESCRIPTION

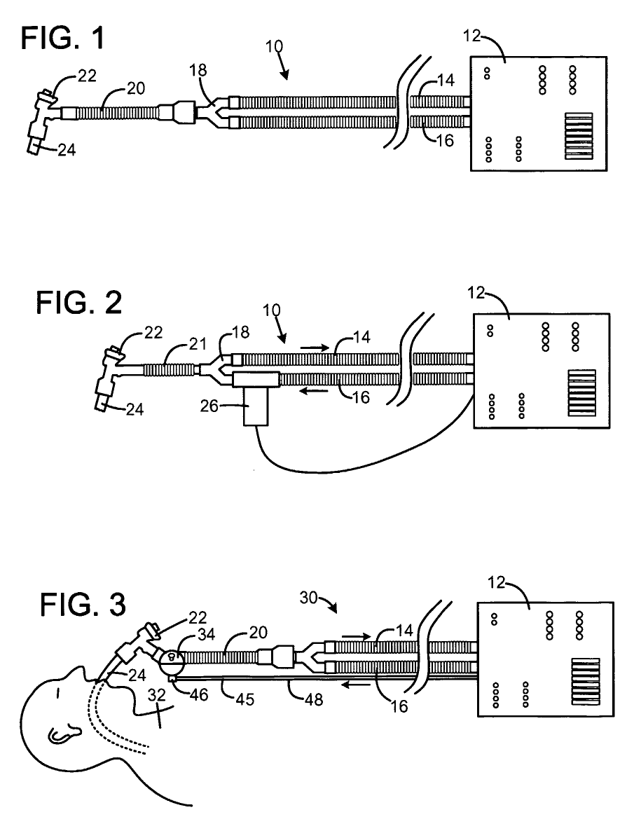

With reference to FIGS. 1-2, a prior art respiration system 10 is shown with a ventilator unit 12 connected to an exhale tube 14 and an inhale tube 16 that are both connected to a Y-section 18. The section 18 is connected to a HME 20 that is connected to a connector unit 22 that may be connected to a flexible tube 24 inserted into the patient. FIG. 2 shows a prior art large nebulizer 26 connected between the inhale tube 16 and the Y-section 18 and the filter 20 has been replaced by an adapter tube 21 to prevent the filter 20 from absorbing the medication administered from the nebulizer 26.

FIG. 3 shows a respirator system 30 of the present invention that includes the ventilator unit 12 connected to the tubes 14, 16 that they are both connected to the Y-section 18 that in turn is connected to the elongate HME 20. Moisture rich air that is exhaled by the patient 32 is absorbed in the filter 20. In this way, dry air that is pumped into the patient 32 from the tube 16 may be moisturized before the air enters the lungs of the patient 32. An important feature of the present invention is that the nebulizer 34 is fully functional even if it is turned up to 45 degrees relative to the horizontal plane and the nebulizer is disposed between and connected to the filter 20 and the connector 22. The connector is connected to the flexible tube 24 that is inserted into the patient 32.

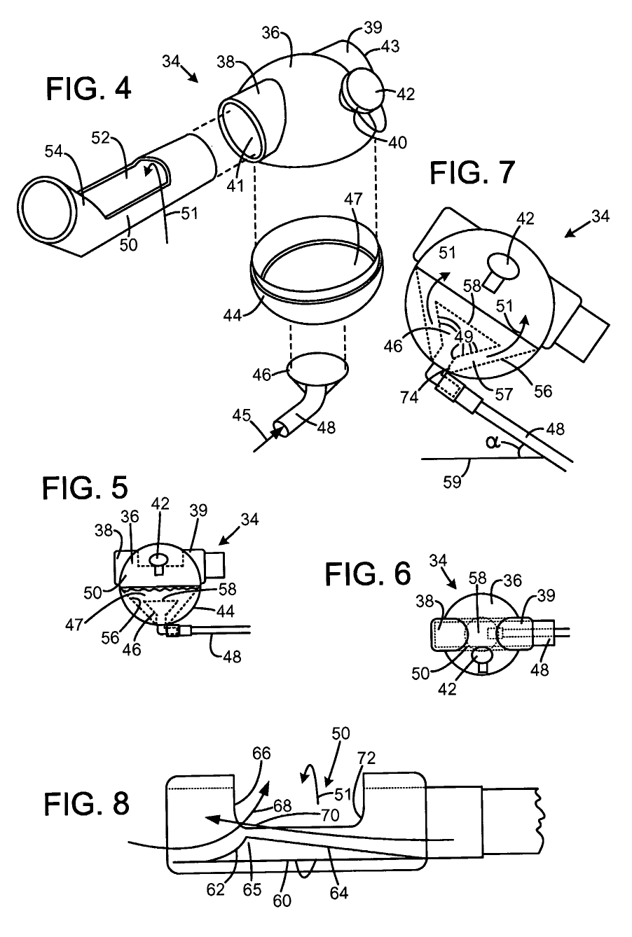

FIG. 4 is an exploded perspective view of the nebulizer 34 and includes an upper semi-spherical housing 36 that has opening segments 38, 39 with openings 41, 43, respectively, defined therein so that the opening 41 is aligned with and in fluid communication with the opening 43. A filler opening 40 with a closable lid 42 is disposed on the upper housing 36. The upper housing 36 is connectable to a lower housing 44 that is connectable to a magnetic valve unit 46 and a return tube 48 that extends back to the ventilator unit 12. When pressurized air 45 is pumped into the tube 48 from the ventilator 12, the air encounters, via the valve 46, a counter pressure of a liquid medication 47 disposed in the lower housing 44. The magnetic valve 46 prevents the medication 47 from going back to the respirator 12 via the tube 48 and the over-pressure in the tube 48 also prevents the medication 47 from flowing back in the tube 48 to the respirator 12. As explained in detailed below, the air 45 causes the liquid medication 47 to aero-sole into aero-soled medication 51 that moves upwardly into the upper housing 36. The aero-sole process of the medication 47 may be synchronized with the pumping rhythm of the ventilator 12 of the inhaling air in the tube 16. A hollow tube 50 is insertable into the openings 41, 43 so that the outer ends of the tube are aligned with the opening segments 38, 39. The tube 50 has an elongate opening 52 defined in an upper wall 54 of the tube 50. In this way, the nebulizer 34 finely distributes the medication added through the opening 40 so that the medication can be effectively inhaled by the patient 32.

FIG. 5 shows a side view of the nebulizer 34 with the tube 50 inserted into the opening segments 38, 39 and the return tube 48 connected to the valve 46 that has many small openings 49 for letting the pressurized air 45 through. Preferably, the lower housing 44 has a conical inner wall 56 so that a channel 57 is formed between the wall 56 and the valve 46.

FIG. 6 shows a top view of the nebulizer 34 with the tube 50 inserted therein. A top section 58 of the valve 46 is shown in the middle of the nebulizer 34.

FIG. 7 shows the nebulizer 34 and the conical inner wall 56. Because the wall 56 is conical and not spherical the liquid medication 47 will always be disposed at the lowest point or bottom 74 of the wall 56. Even if the patient sits up in the bed and the nebulizer 34 is turned at an angle alpha such as 40-45 degrees relative to a horizontal plane 59, the medication 47 is still at the bottom of the wall 56 and exposed to the pressurized air 45 for continuous evaporation of the medication 47.

FIG. 8 is a cross-sectional side view of the tube 50 that has a bottom inner wall 60 with a steeply rising section 62 and a gently rising section 64 that meet adjacent to an end section 65 of the opening 52. Because the tube 50 has the opening 52 facing upwardly, the evaporated medication 51 may rise into the upper housing 36, mix with the turbulent inhale air, move down into the opening 52 and then travel within the tube 50 and the connector 22 into the patient's lungs. Because the tube 50 has a solid bottom wall 60 any secretion in the exhaling air does not fall into the lower housing but continues to travel into the filter 20.

More particularly, the inhaling air flow 68 is exposed to the steeply rising section 62 while the exhaling air flow 70 is exposed to the more gently rising section 64. Because the section 62 is steep, the air becomes turbulent and flows out through the opening 52 and upwardly into the upper housing 36 and is mixed with the aero-soled medication 51 before entering an opening 72 of the tube 50 and into the patient 32. The exhaling air flow is not subject to the steep section, the air flow 70 flows straight through the tube 50 without any significant upward turbulence.

As a result of using the nebulizer 34 of the present invention there is less risk that the lungs collapse and that the SaO2 content is reduced. Also, a bigger portion of the medicine reaches the patient's lungs. There is less risk of infection due to the more closed system and it is possible to use the nebulizer 34 in combination with a closed suction system that is optimal for infection sensitive patients. It is not required to unnecessarily disturb the patient, particularly during the night, when it is time for nebulization.

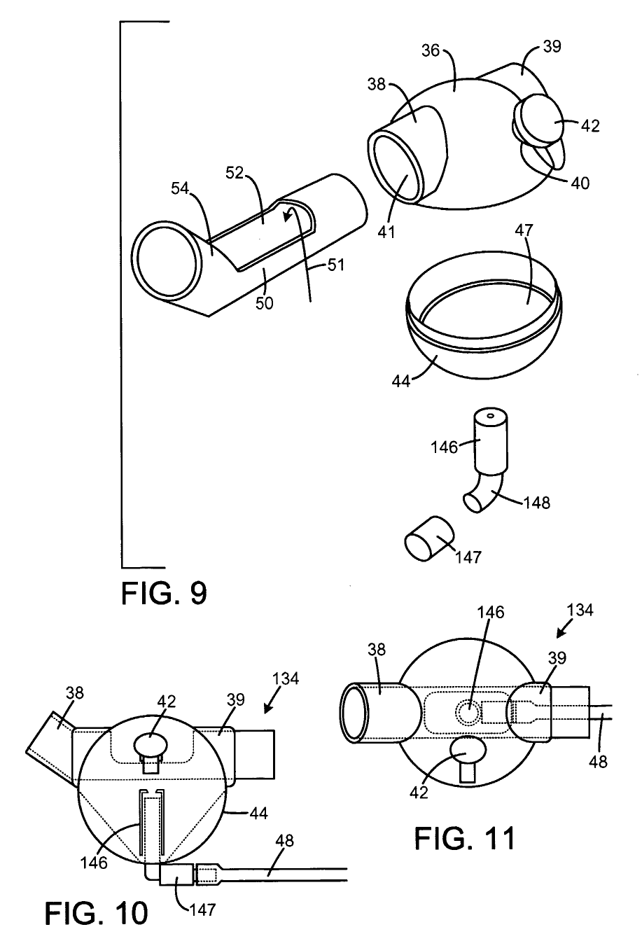

FIGS. 9-11 show an alternative embodiment of a nebulizer 134 that has rectangular unit 146 connected to a hose 148 and a magnetic valve 147. The other features are essentially identical to the nebulizer 34 described above.

While the present invention has been described in accordance with preferred compositions and embodiments, it is to be understood that certain substitutions and alterations may be made thereto without departing from the spirit and scope of the following claims.

1. A method of nebulizing a patient, comprising:
providing a nebulizer (34) having an upper semi-spherical housing (36) in operative engagement with a lower semi-spherical housing (44), the upper housing (36) having openings (41, 43) to receive a tube (50), the tube having an upward opening (52) defined therein;
connecting the nebulizer downstream of a filter (20) of incoming air of an inhale tube (16) and upstream of a connector (22);
adding pressurized air (45) in a tube (48) connected to a bottom (74) of the housing (44);
exposing the air (45) to a liquid medication (47) disposed in the housing (44);
the air (45) aerosolizing the liquid medication (47) into a aerosolized medication (51);
the aero-soled medication (51) mixing with inhaling air provided by a ventilator unit (12) and moisturized by the filter (20);
the aero-soled medication (51) flowing into the opening (52) of the tube (50); and
the aero-soled medication (51) flowing into a patient (32).

2. The method according to claim 1 wherein the method further comprises providing the tube (50) with a bottom inner wall (60) that has a steep section (62) and a gentle section (64).

3. The method according to claim 2 wherein the method further comprises the steep section (62) causing a turbulent air flow (68) that exits through the opening (52).

4. The method according to claim 3 wherein the method further comprises the gentle section (64) causing an air flow (70) that exits through an opening (66) of the tube (50) without escaping through the opening (52).

5. The method according to claim 1 wherein the method further comprises providing the lower housing (44) with a valve (46) having openings (49) to permit the pressurized air (45) into the contact with the liquid medication (47).

6. The method according to claim 1 wherein the method further comprises providing the lower housing (44) with a conical wall (56) to create a channel (57) between the wall (56) and the valve (46).

7. The method according to claim 1 wherein the method further comprises the evaporated medication (51) flowing on an outside of the tube (50) and over the tube (50) before entering into the opening (52).

8. The method according to claim 7 wherein the method further comprises the medication (51) mixing with air in the airflow (68) prior to exiting through an opening (72) of the tube (50).

9. The method according to claim 1 wherein the method further comprises turning the nebulizer (34) up to a 45 degree angle alpha relative to a horizontal plane while permitting the pressurized air (45) to encounter the liquid medication (47).

10. The method according to claim 9 wherein the method further comprises providing the upper housing (36) with an opening (40) for receiving the liquid medication (47).
